(19) United States
(12) Patent Application Publication
(10) Pub. No.: US 2010/0221334 A1
(43) Pub. Date: Sep. 2, 2010

(54) COMPOSITIONS INCLUDING LEUKOTRIENE ANTAGONISTS AND NSAIDS AND METHODS OF USING THE SAME

(76) Inventor: Mitchell P. Fink, Pittsburgh, PA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 12/669,030

(22) PCT Filed: Jul. 18, 2008
(86) PCT No.: PCT/US08/70401

§ 371 Date: May 14, 2010

Related U.S. Application Data
(60) Provisional application No. 60/950,646, filed on Jul. 19, 2007.

(51) Int. Cl. A61K 31/216; A61K 31/381; A61K 9/20; A61K 9/28; A61P 29/00

(52) U.S. Cl. 424/465; 514/510; 514/443; 424/474

(57) ABSTRACT

Pharmaceutical compositions containing non-steroidal anti-inflammatory drugs (NSAIDs) and modified NSAIDs and leukotriene antagonists and methods for using such pharmaceutical compositions are provided herein. In a preffered embodiment naproxene 2-(methanesulfonyl)-ethyl ester is used in combination with Zileuton.

### CROSS-REFERENCE TO RELATED APPLICATIONS

This application claims priority from U.S. Provisional Application No. 60/950,646 entitled “Zileuton and NSAID Compositions and Methods of Using the Same” filed Jul. 19, 2007, the contents of which are hereby incorporated by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

It is well recognized that aspirin and other NSAIDs exert their pharmacological effects through the non-selective inhibition of cyclooxygenase (COX) enzymes, thereby blocking prostaglandin synthesis. There are two types of COX enzymes, namely COX1 and COX2. COX1 is expressed constitutively in many tissues, including the stomach, kidney, and platelets, whereas COX2 is expressed only at the site of inflammation. The prostagladins derived from COX1 are responsible for many of the physiological effects, including maintenance of gastric mucosal integrity. Many attempts have been made to develop NSAIDs that only inhibit COX2, without impacting the activity of COX1. There are several NSAIDs, for example, rofecoxib and celecoxib, that show marked selectivity for COX2. These drugs appear to have reduced gastrointestinal toxicity relative to other NSAIDs. However, the physiological functions of COX1 and COX2 are not always well defined. Thus, there is a possibility that prostaglandins produced as a result of COX1 expression may also contribute to inflammation, pain and fever. On the other hand, prostaglandins produced by COX2 have been shown to play important physiological functions, including the initiation and maintenance of labor and in the regulation of bone resorption. Thus, inhibition of this pathway may not always be beneficial. Considering these points, highly selective COX2 inhibitors have been known to produce cardiovascular side effects and may produce additional side effects above and beyond those observed with standard NSAIDs, and therefore, such inhibitors may not be highly desirable.

Leukotrienes are mediators which belong to the group of eicosanoids. They are derivatives of arachidonic acid, a fatty acid which is a constituent of membrane phospholipids. Leukotrienes are formed from arachidonic acid via 5-lipoxygenase (5-LOX). At the present time, only the pathogenetically relevant role of the so-called cysteinyl-leukotrienes, to which LTC₄, LTD₄ and LTE₄ belong, has been confirmed. The action of the leukotrienes can take place due to occupation of their receptors or by inhibition of their synthesis. In addition to the inhibition of 5-lipoxygenase, the inhibition of a 5-lipoxygenase-activating protein (hereinafter also referred to as “FLAP”) can also lead to decreased synthesis of leukotrienes.

Among the numerous LT antagonists, a few, such as zafirlukast, montelukast, pranlukast, etc. are currently used therapeutically in the treatment of bronchial asthma. Zileuton is marketed as a 5-LOX inhibitors. The so-called FLAP inhibitors include, for example, MK-591, Bay 1005, which are still in the clinical testing phase.

### BRIEF SUMMARY OF THE INVENTION

Various embodiments of the invention are directed to pharmaceutical compositions including a therapeutically effective amount of a modified non-steroidal anti-inflammatory drug (NSAID) of formula:

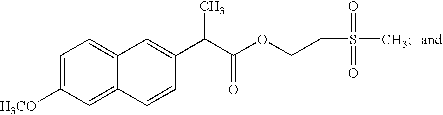

a therapeutically effective amount of one or more leukotriene antagonist, and in some embodiments, the modified non-steroidal anti-inflammatory drug (NSAID) may be of formula:

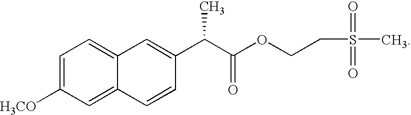

In certain embodiments, the leukotriene antagonist may be a 5-lipoxygenase inhibitor, and in other embodiments, the leukotriene antagonist may be zileuton. In particular embodiments, the pharmaceutical composition may include one or more pharmaceutically acceptable carriers or excipients.

In some embodiments, the composition may be formulated for oral administration, and in particular embodiments, the pharmaceutical composition may be formulated as a tablet. In other embodiments, the modified non-steroidal anti-inflammatory drug (NSAID) and the one or more leukotriene antagonist may be provided in the same dosage unit. In still other embodiments, the modified non-steroidal anti-inflammatory drug (NSAID) and the leukotriene antagonist may be separated by a layer of one or more excipients, and in certain embodiments, the tablet may further include an enteric coating. In yet other embodiments, the modified non-steroidal anti-inflammatory drug (NSAID) and the one or more leukotriene antagonist may be provided in separate dosage units.

Certain embodiments of the invention are directed to pharmaceutical compositions including a therapeutically effective amount of a modified non-steroidal anti-inflammatory drugs (NSAIDs) of formula:

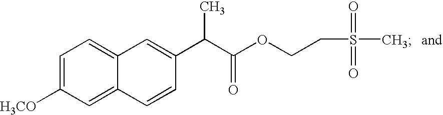

a therapeutically effective amount of zileuton.

Various other embodiments of the invention are directed to methods for treating or preventing pain, inflammation or combination thereof including the steps administering to a subject in need of treatment a pharmaceutical composition which contains a therapeutically effective amount of modified non-steroidal anti-inflammatory drug (NSAID) of formula:

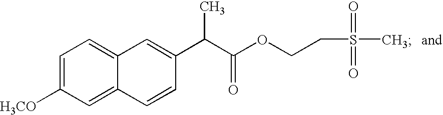

one or more leukotriene antagonists. In some embodiments, the pharmaceutical composition may contain the modified non-steroidal anti-inflammatory drug (NSAID) and the one or more leukotriene antagonists in a single dosage unit, and in other embodiments, the step of administering may include administering the modified non-steroidal anti-inflammatory drug (NSAID) and administering the one or more leukotriene antagonists separately. In certain embodiment, the modified non-steroidal anti-inflammatory drugs (NSAIDs) may be of formula:

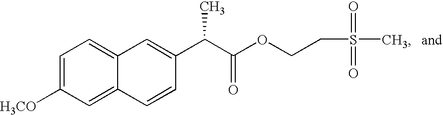

in some embodiments, the leukotriene antagonist may be a 5-lipoxygenase inhibitor. In particular embodiments, the leukotriene antagonist may be zileuton. The pain, inflammation or combination thereof may be associated with any malady including, but not limited to, headache, osteoarthritis and rheumatoid arthritis.

Some embodiments of the invention are directed to methods for treating or preventing pain, inflammation or combination thereof including administering to a subject in need of treatment a pharmaceutical composition containing a compound of formula:

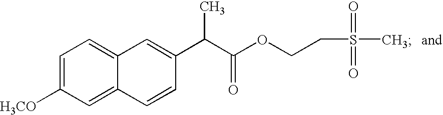

zileuton.

### DESCRIPTION OF DRAWINGS

Not applicable

### DETAILED DESCRIPTION

Before the present formulations, compositions, and methods are described, it is to be understood that this invention is not limited to the particular processes, compositions, formulations, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. All publications mentioned herein are incorporated by reference in their entirety. Nothing herein is to be construed as an admission that the invention is not entitled to antedate such disclosure by virtue of prior invention.

As used herein and in the appended claims, the singular forms “a”, “an”, and “the” include plural reference unless the context clearly dictates otherwise. Thus, for example, reference to a “compound” is a reference to one or more compounds, a class or genus of compounds and any equivalents thereof known to those skilled in the art.

As used herein, the term “about” means plus or minus 10% of the numerical value of the number with which it is being used. Therefore, about 50% means in the range of 45%-55%.

“Administering” as used herein in conjunction with a therapeutic means to administer a therapeutic directly into or onto a target tissue or to administer a therapeutic to a patient whereby the therapeutic positively impacts the tissue to which it is targeted. Thus, as used herein, the term “administering,” can include, but is not limited to, providing a compound or composition described herein into or onto the target tissue; providing such compounds or compositions systemically to a patient by, for example, intravenous injection or oral administration.

The term “animal” as used herein includes, but is not limited to, humans and non-human vertebrates such as wild, domestic and farm animals.

The term “improves” is used to convey that the compounds and/or compositions of the invention enhance the appearance, form, characteristics and/or the physical attributes of a tissue or subject to which it is being provided, applied or administered.

The term “inhibiting” includes the blocking of one or more of a metabolic process, chemical reaction, activity or function of a target protein, tissue or organ and the like by administration of a compound or composition of the invention, or administration of a compound to prevent the onset of the symptoms, alleviating the symptoms, or eliminating the disease, condition or disorder.

By “pharmaceutically acceptable”, it is meant the carrier, diluent or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. For example, in part, embodiments of the present invention are directed to the “therapeutic” treatment of pain and/or inflammation.

A “therapeutically effective amount” or “effective amount” of a composition is a predetermined amount calculated to achieve the desired effect. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated.

The terms “treat,” “treated,” or “treating” as used herein refer to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

As employed herein, “hydrocarbyl” embraces alkyl, substituted alkyl, oxyalkyl, substituted oxyalkyl, cycloalkyl, substituted cycloalkyl, alkenyl, substituted alkenyl, alkynyl, substituted alkynyl, monocyclic heterocylic, substituted monocyclic heterocyclic, monocyclic aromatic, monosubstituted monocyclic aromatic, or the like.

As employed herein, “alkyl” refers to hydrocarbyl radicals having 1 up to 20 carbon atoms, preferably 2-10 carbon atoms; and “substituted alkyl” comprises alkyl groups further bearing one or more substituents selected from hydroxy, alkoxy (of a lower alkyl group), mercapto (of a lower alkyl group), cycloalkyl, substituted cycloalkyl, heterocyclic, substituted heterocyclic, aryl, substituted aryl, heteroaryl, substituted heteroaryl, aryloxy, substituted aryloxy, halogen, trifluoromethyl, cyano, nitro, nitrosothiol (—SNO), nitrate, nitrous acid ester, nitrone, nitrite, nitric acid ester, nitroglyceryl, S-nitrosocysteinyl, S-nitrosoglutathionyl, oxime, N-hydroxylguanidinyl, amino, amido, —C(O)H, acyl, oxyacyl, carboxyl, carbamate, sulfonyl, sulfinyl, sulfonamide, sulfuryl, and the like.

As employed herein, “oxyalkyl” refers to the moiety —O-alkyl-, wherein alkyl is as defined above, and “substituted oxyalkyl” refers to oxyalkyl groups further bearing one or more substituents as set forth above

As employed herein, “cycloalkyl” refers to cyclic ring-containing groups containing in the range of about 3 up to 8 carbon atoms, and “substituted cycloalkyl” refers to cycloalkyl groups further bearing one or more substituents as set forth above.

As employed herein, “heterocyclic” refers to cyclic or ring-containing groups containing one or more heteroatoms, such as, N, O, S, and the like, as part of the ring structure, and having in the range of 3 up to 14 carbon atoms and “substituted heterocyclic” refers to heterocyclic groups further bearing one or more substituents as set forth above.

As employed herein, “alkenyl” refers to straight or branched chain hydrocarbyl groups having at least one carbon-carbon double bond, and having in the range of about 2 up to 12 carbon atoms, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, and “substituted alkynyl” refers to alkynylene groups further bearing one or more substituents as set forth above.

As employed herein, “monocyclic aromatic” refers to aromatic groups having in the range of 5 up to 7 carbon atoms and “monosubstituted monocyclic aromatic” refers to aromatic groups further bearing one of the substituents set forth above.

As employed herein, “alkylene” refers to divalent hydrocarbyl radicals having 1 up to 20 carbon atoms, preferably 2-10 carbon atoms; and “substituted alkylene” comprises alkylene groups further bearing one or more substituents as set forth above.

As employed herein, “cycloalkylene” refers to cyclic ring-containing groups containing in the range of about 3 up to 8 carbon atoms, and “substituted cycloalkylene” refers to cycloalkylene groups further bearing one or more substituents as set forth above.

As employed herein, “oxyalkylene” refers to the moiety —O-alkylene-, wherein alkylene is as defined above, and “substituted oxyalkylene” refers to oxyalkylene groups further bearing one or more substituents as set forth above.

As employed herein, “alkenylene” refers to divalent, straight or branched chain hydrocarbyl groups having at least one carbon-carbon double bond, and having in the range of about 2 up to 12 carbon atoms, and “substituted alkenylene” refers to alkenylene groups further bearing one or more substituents as set forth above.

As employed herein, “alkynylene” refers to divalent straight or branched chain hydrocarbyl groups having at least one carbon-carbon triple bond, and having in the range of about 2 up to 12 carbon atoms, and “substituted alkynylene” refers to alkynylene groups further bearing one or more substituents as set forth above.

As employed herein, “arylene” refers to divalent aromatic groups having in the range of 6 up to 14 carbon atoms and “substituted arylene” refers to arylene groups further bearing one or more substituents as set forth above.

As employed herein, “alkylarylene” refers to alkyl-substituted arylene groups and “substituted alkylarylene” refers to alkylarylene groups further bearing one or more substituents as set forth above.

As employed herein, “arylalkylene” refers to aryl-substituted alkylene groups and “substituted arylalkylene” refers to arylalkylene groups further bearing one or more substituents as set forth above.

As employed herein, “arylalkenylene” refers to aryl-substituted alkenylene groups and “substituted arylalkenylene” refers to arylalkenylene groups further bearing one or more substituents as set forth above.

As employed herein, “arylalkynylene” refers to aryl-substituted alkynylene groups and “substituted arylalkynylene” refers to arylalkynylene groups further bearing one or more substituents as set forth above.

It will be understood that, for the purposes of this disclosure, reference to an NSAID, leukotriene antagonist, 5-lipoxygenase inhibitor or analgesic agent will include all of the common forms of these compounds and their pharmaceutically acceptable salts.

Embodiments of the invention described herein are generally directed to pharmaceutical compositions including a cyclooxygenase enzyme (COX) inhibitor and a leukotriene inhibitor and methods for treating a disease using such pharmaceutical compositions.

COX inhibitors are well known and utilized in the pharmaceutical arts, and any COX inhibitor may be utilized in various embodiments of the invention. For example, in some embodiments, the COX inhibitor may be a non-steroidal anti-inflammatory drug (NSAID) or a prodrug thereof such as, but not limited to, acetaminophen, aspirin, ibuprofen, choline magnesium salicylate, choline salicylate, diclofenac, diflunisal, etodolac, fenprofen calcium, fluorobiprofen, indomethacin, ketoprofen, carprofen, indoprofen, ketorolac tromethamine, magnesium salicylate, meclofenamate sodium, mefenamic acid, oxaprozin, piroxicam, sodium salicylate, sulindac, tolmetin, meloxicam, nabumetone, naproxen, lornoxicam, nimesulide, indoprofen, remifenzone, salsalate, tiaprofenic acid, flosulide and the like and combinations thereof. In other embodiments, the NSAID may be a cyclooxygenase-2 inhibitors (COX-2) inhibitor including, but not limited to, celecoxib, rofecoxib, meloxicam, piroxicam, valdecoxib, parecoxib, etoricoxib, CS-502, JTE-522, L-745,337 and NS398 and combinations thereof. In particular embodiments, the NSAID may be naproxen.

In other embodiments, the NSAID may be an NSAID that has been modified to form a prodrug, sustained release or “long acting” form of the NSAID as is known in the art. As used herein, the term “long acting” refers to an NSAID having a pharmacokinetic half-life of at least about 2 hours, at least about 4 hours and in particular embodiments, at least about 8 to 14 hours. For example, long-acting NSAIDs include, but are not limited to, flurbiprofen with a half-life of about 6 hours; ketoprofen with a half-life of about 2 to 4 hours; naproxen or naproxen sodium with half-lives of about 12 to 15 hours and about 12 to 13 hours respectively; oxaprozin with a half life of about 42 to 50 hours; etodolac with a half life of about 7 hours; indomethacin with a half life of about 4 to 6 hours; ketorolac with a half-life of up to about 8-9 hours, nabumetone with a half-life of about 22 to 30 hours; mefenamic acid with a half-life of up to about 4 hours; and piroxicam with a half-life of about 4 to 6 hours. If an NSAID does not naturally have a half-life sufficient to be long acting, it can, if desired, be made long acting by the way in which it is formulated. For example, NSAIDs such as acetaminophen and aspirin may be formulated in a manner to increase their half-life or duration of action. Methods for making appropriate formulations are well known in the art (see Remington's Pharmaceutical Sciences, 16th ed., A. Oslo editor, Easton, Pa. (1980)).

In other embodiments, the NSAID may be modified to produce a long acting compound and/or to reduce side effects associated with administration of NSAIDs. For example, in some embodiments, the NSAID may be a compound of general formula I:

X-L-Z (I)

where:

X=a (NSAID),

L=an optional linker/spacer, and

Z=a sulfur-containing functional group.

Examples of such compounds and methods for using such compounds can be found in U.S. Pat. No. 6,355,666, U.S. Pat. No. 6,429,223 and U.S. Publication No. 2003/0220468 which are hereby incorporated by reference in their entireties. Any of the NSAIDs listed above may be used as the NSAID (X) in such embodiments, and in particular embodiments, the NSAID employed may be naproxen, aspirin, ibuprofen, flurbiprofen, indomethacin, ketoprofen, carprofen, and the like. Sulfur-containing functional groups (Z) contemplated by the present invention include, for example, sulfonate, reverse sulfonate, sulfonamide, reverse sulfonamide, sulfone, sulfoxide, sulfinate, reverse sulfinate, sulfide, and the like, and in some embodiments, the sulfur-containing functional group may be a sulfonate or reverse sulfonate or an optionally substituted aromatic sulfonate such as tosylate or brosylate. Other exemplary sulfur-containing functional groups include, —SCH₃, —S(O)CH₃, —S(O₂)CH₃, —S(O₂)C₆H₅, —S(O₂)C₆H₄NO₂, and the like.

The NSAID and sulfur-containing function group of compositions of the invention may be directly or indirectly covalently attached employing a variety of linkers (L) such as, for example, C₁₋₂₀ alkylene, substituted C₁₋₂₀ alkylene, C₂₋₁₂ alkenylene, alkynylene or oxalkylene, substituted C₂₋₁₂ alkenylene, alkynylene or oxalkylene, C₃₋₁₂ cycloalkylene or heterocycloalkylene, substituted C₃₋₁₂ cycloalkylene or heterocycloalkylene, C₆₋₁₄ arylene or heteroarylene, substituted C₆₋₁₄ arylene or heteroarylene, ester linkages, disulfide linkages, amide linkages, immine linkages, enamine linkages, ether linkages, thioether linkages, imide linkages, sulfate ester linkages, sulfonate ester linkages, sulfone linkages, sulfonamide linkages, phosphate ester linkages, carbonate linkages, O-glycosidic linkages, S-glycosidic linkages, and the like. Standard synthetic techniques well known by those skilled in the art may be utilized to create a vast number of such linkages either by direct reaction of the starting materials, or by incorporating a suitable functional group on the starting material, followed by coupling of the reactants.

In certain exemplary embodiments, a modified NSAID may be of general formula II:

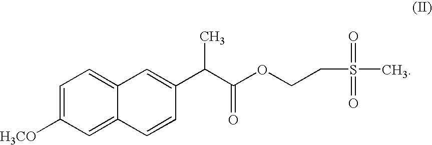

Other modified NSAIDs useful in embodiments of the invention include, for example, those set forth and described in U.S. Pat. Nos. 5,516,789, 5,220,059, 6,057,347, 6,297,260, 6,306,842, 6,407,135, 6,620,813 and 6,710,086, U.S. Publication Nos. 2005/0129774 and 2005/0222243, PCT Publication Nos. WO 91/08744, WO 95/04528, WO 95/07104 and WO 96/22780, EP 0814839 and GB 2321455 the disclosures of each of which are hereby incorporated by reference in their entireties.

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

Leukotriene antagonists are well known in the art and any compound having a therapeutic activity which inhibits either the activity or synthesis of leukotrienes may be used in embodiments of the invention. For example, compounds have been isolated which inhibit leukotriene activity such as, but not limited to, montelukast, zafirlukast, pranlukast, iralukast, pobilukast, SKB-106,203, Bay-1005, L663536 and zileuton. In some embodiments, the leukotriene antagonist may inhibit leukotriene activity by inhibiting leukotriene signaling by, for example, inhibiting binding of leukotriene to an active site. In other embodiments, the leukotriene inhibitor may be a 5-lipoxygenase inhibitor such as zileuton, BAY-1005 or L663536 which inhibit 5-lipoxygenase function thereby reducing the formation of leukotrienes including LTB₄, LTC₄, LTD₄ and LTF₄. Further details regarding zileuton and L663536 can be found in, for example, U.S. Pat. No. 4,873,259 and U.S. Pat. Nos. 7,132,441, 7,371,889 and 7,378,442 each of which are hereby incorporated by reference in their entireties.

Various embodiments of the invention are directed to compositions including an NSAID or modified NSAID and a leukotriene antagonist. In some embodiments, the NSAID, modified NSAID and leukotriene antagonist may be provided in a single unit dosage such as a tablet capsule, liquid suspension or solution, dispersion and so on containing a therapeutically effective amount of both components. In other embodiments, the NSAID, modified NSAID and leukotriene antagonist may be provided in separate unit dosages, one containing a therapeutically effective amount of the NSAID or modified NSAID and the other containing a therapeutically effective amount of the leukotriene antagonist.

Without wishing to be bound by theory, the NSAID or modified NSAID and the leukotriene antagonist of such embodiments may be effective over a wide dosage range. A therapeutically effective amount of a compound of this invention is typically an amount such that when it is administered in a physiologically tolerable excipient composition, it is sufficient to achieve an effective systemic concentration or local concentration in the tissue. For example, an effective amount of NSAID or modified NSAID may be an amount that inhibits, blocks or prevents pain and inflammation, and an effective amount of a leukotriene antagonist such as a 5-lipoxygenase inhibitor may be an amount that decreases the formation of LTB₄, LTC₄, LTD₄, LTF₄ and other leukotrienes.

In certain embodiments, the modified NSAID may be naproxen 2-(methanesulfonyl)ethyl ester as disclosed in U.S. Pat. No. 6,355,666 (application Ser. No. 09/602,688), which is hereby incorporated by reference in its entirety, as Compound 50 and a method of making Compound 50 is disclosed in Example 17. Naproxen 2-(methanesulfonyl)ethyl ester is also called (S)-(+)-2-(6-methoxy-2-naphthyl)propionic acid 2-(methanesulfonyl)ethyl ester. The structure of racemic 2-(6-methoxy-2-naphthyl)propionic acid 2-(methanesulfonyl)ethyl ester is:

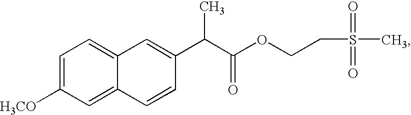

and the structure of (S)-(+)-2-(6-methoxy-2-naphthyl)propionic acid 2-(methanesulfonyl)ethyl ester (naproxen 2-(methylsulfonyl)ethyl ester) is:

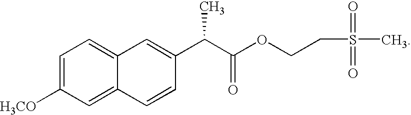

A therapeutically effective amount of the NSAID or modified NSAID and the leukotriene antagonist may vary and dosages for each component may independently fall within the range of from about 0.001 mg/kg of body weight to about 10 mg/kg of body weight. It will be appreciated by the skilled artisan that a therapeutically effective amount can readily be determined by the physician who may take into consideration circumstances including, for example, the condition to be treated, the choice of compound administered and the route of administration. Therefore, a therapeutically effective amount may be greater or less than the above specified dosage range. It is expected that a skilled pharmacologist may adjust the amount of drug in a pharmaceutical composition or administered to a patient based upon standard techniques well known in the art. Nevertheless, the following typical daily dosage guidelines are provided as exemplary embodiments: indomethacin up to about 150 mg per day; aspirin from 500 mg to about 10 g; ibuprofen up to about 3200 mg; flurbiprofen from about 100 mg to 500 mg; ketoprofen from about 100 mg to 500 mg; naproxen from about 100 mg to 1250 mg; oxaprozin from less than about 1200 mg to about 1800 mg or 26 mg/kg; etodolac up to about 1200 mg; ketorolac up to 50 mg; nabumetone from about 1500 mg to about 2000 mg; mefenamic from about 1 mg to about 1000 mg; lornoxicam from about 8 mg to 16 mg for pain, and for arthritis about 12 mg; celecoxib from about 100 mg to about 500 mg; piroxicam from about 10 mg to 20 mg; rofecoxib about 50 mg; meloxicam from about 7.5 mg to about 15 mg; and valdecoxib from about 5 mg to about 10 mg for arthritis and about 40 mg for dysmenorrheal;

Leukotriene antagonists such as, for example, 5-lipoxygenase inhibitors may be administered in a daily dose of from about 200 mg to about 3,000 mg and in some embodiments, from about 500 mg to about 2,400 mg. For example, zileuton tablets and zileuton extended release tablets may include about a 600 mg dose.

As is known in the art, daily doses of NSAIDs, leukotriene antagonists and combinations thereof, may be delivered in divided doses. For example, an NSAID and/or leukotriene antagonist may be prepared as a tablet or capsule containing, for example, 50 mg, 100 mg, 200 mg, 300 mg, 400 mg, 600 mg, 800 mg or 1000 mg that are administered 2, 3, 4, 5 or 6 times per day until an adequate daily dosage is achieved. For example, naproxen may be prepared as tablets or capsules containing from 250 to 500 mg of naproxen, or for naproxen sodium, tablets or capsules may contain from about 275 to about 550 mg of naproxen sodium. Initial doses of from about 100 mg to about 1250 mg and in some embodiments, about 350 mg to about 800 mg may be administered daily in 2 or more doses.

Further embodiments of the invention are directed to pharmaceutical compositions including an NSAID or modified NSAID and a leukotriene antagonist and one or more pharmaceutically acceptable excipient and/or carriers, and in particular embodiments, the NSAID may be naproxen and the leukotriene antagonist may be a 5-lipoxygenase inhibitor such as zileuton. In some embodiments, such pharmaceutical compositions may further include one or more auxiliary agents such as, for example, lubricants, preservatives, disintegrants, stabilizers, wetting agents, emulsifiers, salts, buffers, coloring agents, flavoring agents, or aromatic substances. Such pharmaceutical compositions may be formulated in any way known in the art. For example, in some embodiments, such pharmaceutical compositions may be formulates as tablets, capsules, dragees, liquids, dispersions, suspensions, and the like that can be made in accordance with methods that are standard in the art (see Remington's Pharmaceutical Sciences, 16th ea., A Oslo editor, Easton, Pa. (1980)).

In such embodiments, pharmaceutical compositions may include an NSAID and/or a leukotriene in an amount effective to reduce, eliminate or prevent pain or inflammation. For example, a pharmaceutical composition encompassed by the invention may include from about 10 mg to about 2,000 mg of an NSAID such as aspirin, acetaminophen, ibuprofen, flurbiprofen, ketoprofen, naproxen, oxaprozin, etodolac, indomethacin, ketorolac, lornoxicam, nabumetone, or diclofenac. In some embodiments, such pharmaceutical compositions may include from about 50 mg to about 1500 mg or from about 200 mg to about 600 mg of naproxen. Similarly, a pharmaceutical composition encompassed by the invention may include from about 10 mg to about 2,000 mg or, in some embodiments, about 500 mg to about 1,000 mg of a leukotriene antagonist such as a 5-lipoxygenase inhibitor, for example, zileuton. Without wishing to be bound by theory, the combined effect of the NSAID, modified NSAID and leukotriene antagonist may allow for a therapeutically effective amount of the constituents of the pharmaceutical composition to be reduced to below a standard therapeutically effective amount. Thus, in some embodiments, the one or more NSAIDs, modified NSAIDs and/or leukotriene antagonists of the pharmaceutical compositions of the invention may include a therapeutically effective amount of each component that is less than the standard therapeutically effective amount.

Depending on the mode of delivery employed, the modified NSAIDs contemplated for use herein can be delivered in a variety of pharmaceutically acceptable forms. For example, the invention modified NSAIDs and/or acid inhibitor can be delivered in the form of a solid, solution, emulsion, dispersion, micelle, liposome, and the like. Thus, some embodiments of the invention include pharmaceutical compositions containing an NSAID or modified NSAIDs and/or a leukotriene antagonist and a pharmaceutically acceptable carrier or excipient rendering the composition suitable for oral delivery, transdermal delivery, intravenous delivery, intramuscular delivery, topical delivery, nasal delivery, and the like.

In various embodiments, the pharmaceutical compositions of the invention can be used in the form of a solid, a solution, an emulsion, a dispersion, a micelle, a liposome, and the like, wherein the resulting composition contains one or more NSAIDs or modified NSAIDs and/or leukotriene antagonists in an admixture with an organic or inorganic carrier or excipient suitable for enteral or parenteral applications. The one or more NSAIDs, modified NSAIDs and/or leukotriene antagonists may be compounded, for example, with a non-toxic, pharmaceutically acceptable carrier suitable for forming tablets, pellets, capsules, suppositories, solutions, emulsions, suspensions, and any other dosage form. Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; containing an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

More specifically, in some embodiments, pharmaceutical compositions may include one or more NSAIDs, modified NSAIDs and/or leukotriene antagonists in a form suitable for oral administration, for example, tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions, and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically accepable and palatable preparations.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

In certain embodiments, the one or more NSAIDs, modified NSAIDs and/or leukotriene antagonists may be formed into a tablet including one or more excipients or carriers. For example, in some embodiments, several excipients may be used such as (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets of various embodiments may be uncoated or they may be coated by known techniques to delay disintegration and absorption in the gastrointestinal tract. For example, a material such as glyceryl monostearate or glyceryl distearate may be used to coat the tablet. In other embodiments, the tablets of the invention may be coated by the techniques described in the U.S. Pat. Nos. 4,256,108; 4,160,452; and 4,265,874, to form osmotic therapeutic tablets. In some embodiments, the NSAID may be granulated by methods such as slugging, low- or high-shear granulation, wet granulation, or fluidized-bed granulation. Of these processes, slugging generally produced tablets of less hardness and greater friability. Low-shear granulation, high-shear granulation, wet granulation and fluidized-bed granulation generally produce harder, less friable tablets.

Pharmaceutical preparations which can be used orally include, but are not limited to, push-fit capsules made of gelatin, as well as soft, sealed capsules made of gelatin and a plasticizer, such as glycerol, sorbitol, calcium carbonate, calcium phosphate or kaolin. The push-fit capsules can contain the active ingredients in admixture with filler such as, for example, lactose and the like, binders such as, for example, starches and the like, and/or lubricants such as, for example, talc, magnesium stearate and the like and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

In other embodiments, the pharmaceutical compositions of the invention may include an enteric coating which may be applied onto a core or onto a barrier layer of the core. The enteric coating materials may be dissolved or dispersed in organic or aqueous solvents and may include one or more of the following materials: methacrylic acid copolymers, shellac, hydroxypropylmethcellulose phthalate, polyvinyl acetate phthalate, hydroxypropylmethylcellulose trimellitate, carboxymethylethyl-cellulose, cellulose acetate phthalate or other suitable enteric coating polymer(s). The pH at which the enteric coat will dissolve can be controlled by the polymer or combination of polymers selected and/or ratio of pendant groups. For example, dissolution characteristics of the polymer film can be altered by the ratio of free carboxyl groups to ester groups. Enteric coatings may also contain pharmaceutically acceptable plasticizers such as triethyl citrate, dibutyl phthalate, triacetin, polyethylene glycols, polysorbates or other plasticizers. Additives such as dispersants, colorants, anti-adhering and anti-foaming agents may also be included.

In still other embodiments, the pharmaceutical compositions may be in the form of a sterile injectable form such as a suspension. Such suspensions may be formulated according to known methods using suitable dispersing or wetting agents and/or suspending agents. The sterile injectable preparation may also be a sterile injectable solution or suspension in a pharmaceutically acceptable diluent or solvent, for example, sterile water or saline, alcohols, 1,3-butanediol, sterile or fixed oils. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can also be incorporated as required.

The one or more NSAIDs, modified NSAIDs and/or leukotriene antagonists of the invention can be formulated for parenteral administration by injection, bolus injection or continuous infusion. For example, the compositions of the invention can be administered by continuous infusion subcutaneously over a period of about 15 minutes to about 24 hours. Formulations for injection can be presented in unit dosage form or in multi-dose containers, with an added preservative. The compositions can take such forms as suspensions, solutions or emulsions in oily or aqueous vehicles, and can contain formulatory agents such as suspending, stabilizing and/or dispersing agents.

In addition to the formulations described previously, the compounds of the present invention can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, for example, gelatin for use in an inhaler or insufflator can be formulated containing a powder mix of the compound and a suitable powder base such as lactose or starch.

In yet other embodiments, the one or more NSAIDs, modified NSAIDs and/or leukotriene antagonists may be formulated for use as suppositories for rectal administration of the drug. These compositions may be prepared by mixing one or more NSAIDs, modified NSAIDs and/or leukotriene antagonists with a suitable non-irritating excipient, such as cocoa butter, synthetic glyceride esters of polyethylene glycols, which are solid at ordinary temperatures, but liquify and/or dissolve in the rectal cavity to release the drug

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein. For example, in some embodiments, the pharmaceutical compositions of the invention may include an acid inhibitor in an amount sufficient to raise the gastric pH of a patient to at least about 3.5, at least about 4 or at least about 5. It is noted that the gastric pH should not generally exceed 7.5. The term “acid inhibitor” refers to agents that inhibit gastric acid secretion and increase gastric pH. In contrast to art teaching against the use of H2 blockers for the prevention of NSAID-associated ulcers (N. Eng J. Med. 340: 1888-1899 (1999)), these agents may also be used in the various embodiments of the compositions and methods of the present invention. Specific H2 blockers that may be used include cimetidine, ranitidine, ebrotidine, pabutidine, lafutidine, loxtidine or famotidine. Other agents that may be effectively used include proton pump inhibitors such as omeprazole, esomeprazole, pantoprazole, lansoprazole or rabeprazole. Such acid inhibitors may be provided in the pharmaceutical composition or in other embodiments, an acid inhibitor may be provided in one or more separate unit dosage forms. The skilled artisan may determine the appropriate amount of acid inhibitor present in the pharmaceutical compositions of the invention.

In certain embodiments, the 5-lipoxygenase inhibitor and NSAID may be delivered as part of a single unit dosage form which provides for the coordinated release of therapeutic agents. For example, in some embodiments, the pharmaceutical compositions of the invention may be formulated as multilayer composition, such as, bi- or multi-layer tablet. In a bilayer configuration, one portion of the tablet may contain the leukotriene antagonist in the required dose along with the appropriate excipients, agents to aid dissolution, lubricants, fillers, etc., and a second portion of the tablet may contain a NSAID or a modified NSAID, in the required dose along with other excipients, dissolution agents, lubricants, fillers, etc. In one exemplary embodiment, a multilayer tablet may have an outer layer of a 5-lipoxygenase inhibitor such as zileuton and an inner core of an NSAID such as naproxen. In another exemplary embodiment, the NSAID layer may be surrounded by a polymeric coating which does not dissolve at a pH of less than 4. In particular embodiments, the multilayer tablet may include a sulfur modified naproxen and a time released zileuton. In other embodiments, the leukotriene antagonist and NSAID may be provided in separate dosage formulations. For example, in one embodiment, a 5-lipoxygenase inhibitor such as zileuton and an NSAID such as naproxen may be provided in separate doses, in two separate tablets. In such embodiments, the NSAID can be administered prior to, simultaneously with or following administration of the 5-lipoxygenase inhibitor.

Further embodiments of the invention include methods for treating a patient for pain, inflammation and/or other conditions by administering an NSAID and a leukotriene antagonist. Generally, such methods include the step of administering to a patient in need of treatment an effective amount of a composition including an NSAID and a leukotriene antagonist. In some embodiments, administering may include administering a single composition including both the NSAID and the leukotriene antagonist, and in other embodiments, administering may include administering a single composition including both components. The method of various embodiments of the invention may be used for any condition in which an NSAID is effective. Diseases and conditions contemplated for treatment in accordance with the present invention include inflammatory and infectious diseases, such as, for example, septic shock, hemorrhagic shock, anaphylactic shock, toxic shock syndrome, ischemia, cerebral ischemia, administration of cytokines, overexpression of cytokines, ulcers, inflammatory bowel disease, such as, ulcerative colitis or Crohn's disease, diabetes, arthritis, rheumatoid arthritis, osteoarthritis, asthma, Alzheimer's disease, Parkinson's disease, multiple sclerosis, cirrhosis, allograft rejection, encephalomyelitis, meningitis, pancreatitis, peritonitis, vasculitis, lymphocytic choriomeningitis, glomerulonephritis, uveitis, ileitis, inflammation (for example, liver inflammation, renal inflammation, and the like), burn, infection (including bacterial, viral, fungal and parasitic infections), hemodialysis, chronic fatigue syndrome, stroke, cancers such as, for example, breast, melanoma, carcinoma, and the like, cardiopulmonary bypass, ischemic/reperfusion injury, gastritis, adult respiratory distress syndrome, cachexia, myocarditis, autoimmune disorders, eczema, psoriasis, heart failure, heart disease, atherosclerosis, dermatitis, urticaria, systemic lupus erythematosus, AIDS, AIDS dementia, chronic neurodegenerative disease, pain, chronic pain, post-surgical pain, priapism, cystic fibrosis, amyotrophic lateral sclerosis, schizophrenia, depression, premenstrual syndrome, anxiety, addiction, headache, migraine, Huntington's disease, epilepsy, neurodegencrative disorders, gastrointestinal motility disorders, obesity, hyperphagia, solid tumors such as, for example, neuroblastoma, malaria, hematologic cancers, myelofibrosis, lung injury, graftversushost disease, head injury, CNS trauma, hepatitis, renal failure, liver disease, chronic hepatitis C, drug-induced lung injury, paraquat, myasthenia gravis (MG), ophthalmic diseases, postangioplasty, restenosis, angina, coronary artery disease, and the like. In certain embodiments, the condition treated using the pharmaceutical compositions of the invention include all forms of headache including migraine headache, acute musculoskeletal pain, ankylosing spondylitis, dysmenorrhoea, myalgias, and neuralgias. In particular embodiments, such methods may be used to treat patients with osteoarthritis or rheumatoid arthritis. Since individual subjects may present a wide variation in severity of symptoms and each drug has its unique therapeutic characteristics, the precise mode of administration and dosage employed for each subject is left to the discretion of the practitioner.

The compounds of the present invention can be administered in the conventional manner by any route where they are active. Administration can be systemic, topical or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, for example, the particular animal treated, age, weight, health, types of concurrent treatment, if any, and frequency of treatments, and can be easily determined by one of skill in the art such as a clinician.

Other embodiments of the invention include methods for increasing compliance in a patient requiring frequent daily dosing of NSAIDs by administering a leukotriene antagonist and an NSAID. As described above, in some embodiments, the leukotriene antagonist and NSAID may be provided in a single pharmaceutical composition, in coordinated unit dosage form, to reduce the total number of individual doses administered during any given period. In other embodiments, the leukotriene antagonist and the NSAID may be provided in separate individual dosage formulations. In certain embodiments, the method for increasing compliance in a patient includes providing a single unit dosage form including both naproxen and zilueton or a sulfur modified form of naproxen and the extended release form of zilueton.

Although the present invention has been described in considerable detail with reference to certain preferred embodiments thereof, other versions are possible. Therefore the spirit and scope of the appended claims should not be limited to the description and the preferred versions contained within this specification.

### Example 1

The following compositions are representative compositions which could be made according to embodiments of the present invention.

A. 200 mg to 600 mg naproxen 2-(methanesulfonyl)ethyl ester and 50 mg zilueton;

B. 200 mg to 600 mg naproxen 2-(methanesulfonyl)ethyl ester and 100 mg zilueton;

C. 200 mg to 600 mg naproxen 2-(methanesulfonyl)ethyl ester and 300 mg zilueton;

D. 200 mg to 600 mg naproxen 2-(methanesulfonyl)ethyl ester and 500 mg zilueton;

E. 200 mg to 600 mg naproxen 2-(methanesulfonyl)ethyl ester and 600 mg zilueton;

F. 200 mg to 600 mg naproxen 2-(methanesulfonyl)ethyl ester and 1200 mg zilueton;

G. 200 mg to 600 mg naproxen 2-(methanesulfonyl)ethyl ester and 1800 mg zilueton;

H. 200 mg to 600 mg naproxen 2-(methanesulfonyl)ethyl ester and 2400 mg zilueton;

Any one of the above compositions could be combined with one or more excipients.

What is claimed is:

1. A pharmaceutical composition comprising:
a therapeutically effective amount of a modified non-steroidal anti-inflammatory drug (NSAID) of formula: 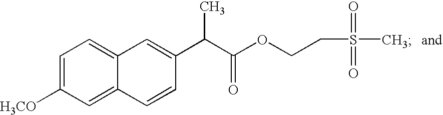
a therapeutically effective amount of one or more leukotriene antagonist.

2. The pharmaceutical composition of claim 1, wherein the modified non-steroidal anti-inflammatory drug (NSAID) is of formula:

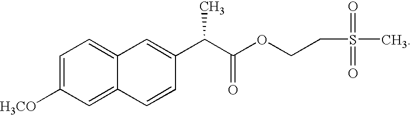

3. The pharmaceutical composition of claim 1, wherein the leukotriene antagonist is a 5-lipoxygenase inhibitor.

4. The pharmaceutical composition of claim 1, wherein the leukotriene antagonist is zileuton.

5. The pharmaceutical composition of claim 1, wherein said composition is formulated for oral administration.

6. The pharmaceutical composition of claim 1, wherein the modified non-steroidal anti-inflammatory drug (NSAID) and the one or more leukotriene antagonist is provided in the same dosage unit.

7. The pharmaceutical composition of claim 1, wherein the pharmaceutical composition is formulated as a tablet.

8. The pharmaceutical composition of claim 7, wherein the modified non-steroidal anti-inflammatory drug (NSAID) and the leukotriene antagonist are separated by a layer of one or more excipients.

9. The pharmaceutical composition of claim 7, wherein the tablet further comprises an enteric coating.

10. The pharmaceutical composition of claim 1, wherein the modified non-steroidal anti-inflammatory drug (NSAID) and the one or more leukotriene antagonist is provided in separate dosage units.

11. The pharmaceutical composition of claim 1, further comprising one or more pharmaceutically acceptable carriers or excipients.

12. A pharmaceutical composition comprising:
a therapeutically effective amount of a modified non-steroidal anti-inflammatory drugs (NSAIDs) of formula: 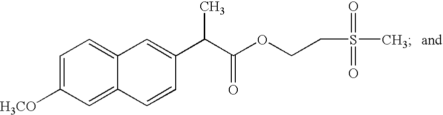
a therapeutically effective amount of zileuton.

13. A method for treating or preventing pain, inflammation or combination thereof comprising administering to a subject in need of treatment a pharmaceutical composition comprising:
a therapeutically effective amount of modified non-steroidal anti-inflammatory drug (NSAID) of formula: 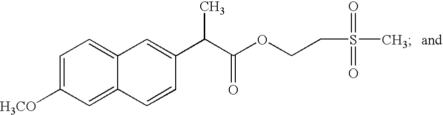
one or more leukotriene antagonists.

14. The method of claim 13, wherein the pharmaceutical composition comprises the modified non-steroidal anti-inflammatory drug (NSAID) and the one or more leukotriene antagonists in a single dosage unit.

15. The method of claim 13, wherein administering comprises administering the modified non-steroidal anti-inflammatory drug (NSAID) and administering the one or more leukotriene antagonists separately.

16. The method of claim 13, wherein the modified non-steroidal anti-inflammatory drugs (NSAIDs) is of formula:

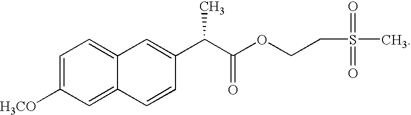

17. The method of claim 13, wherein the leukotriene antagonist is a 5-lipoxygenase inhibitor.

18. The method of claim 13, wherein the leukotriene antagonist is zileuton.

19. The method of claim 13, wherein pain, inflammation or combination thereof is associated with a malady selected from headache, osteoarthritis and rheumatoid arthritis.

20. A method for treating or preventing pain, inflammation or combination thereof comprising administering to a subject in need of treatment a pharmaceutical composition comprising:
a compound of formula: 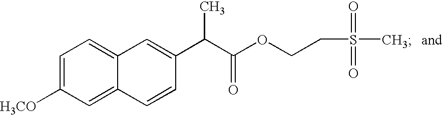
zileuton.
